(19) United States
(12) Patent Application Publication
(10) Pub. No.: US 2012/0027818 A1
(43) Pub. Date: Feb. 2, 2012

(54) BISPHOSPHONATE PRODUCT IN A CYCLOOLEFINIC POLYMER CONTAINER

(76) Inventors: Alexandra Glausch, Weil am Rhein (DE); Rolf Loeffler, Freiburg (DE); Juergen Sigg, Loerrach (DE)

(21) Appl. No.: 13/242,613

(22) Filed: Sep. 23, 2011

Related U.S. Application Data
(63) Continuation of application No. 12/141,676, filed on Jun. 18, 2008.
Application No. 12/141,676 is a continuation of application No. 10/941,229, filed on Sep. 15, 2004, now abandoned.

(51) Int. Cl. A61K 9/00; A61B 19/02; A61P 19/10; A61K 31/675; A61P 19/08

(52) U.S. Cl. 424/400; 514/94; 206/438

(57) ABSTRACT

A pharmaceutical product comprises a container containing a bisphosphonate solution, in which at least the internal surface of the container comprises a plastic material and in which the container is heat sterilisable, and which is in the form of an infusion solution preconcentrate for administration of the bisphosphonate to a patient in need of bisphosphonate treatment.

### CROSS REFERENCE TO RELATED APPLICATION

This application is a continuation application of U.S. application Ser. No. 12/141,676, filed Jun. 18, 2008, which is a continuation of U.S. application Ser. No. 10/641,229, filed Sep. 15, 2004 and currently abandoned, the disclosures of which are expressly incorporated herein by reference in their entirety.

This invention relates to pharmaceutical products and processes for their production, in particular to pharmaceutical products comprising bisphosphonates and to processes for producing such bisphosphonate products.

Bisphosphonates are widely used to inhibit osteoclast activity in a variety of both benign and malignant diseases which involve excessive or inappropriate bone resorption. These pyrophosphate analogs not only reduce the occurrence of skeletal related events but they also provide patients with clinical benefit and improve survival. Bisphosphonates are able to prevent bone resorption in vivo; the therapeutic efficacy of bisphosphonates has been demonstrated in the treatment of osteoporosis, osteopenia, Paget's disease of bone, tumour-induced hypercalcemia (TIH) and, more recently, bone metastases (BM) and multiple myeloma (MM) (for review see Fleisch H 1997 Bisphosphonates clinical. In Bisphosphonates in Bone Disease. From the Laboratory to the Patient. Eds: The Parthenon Publishing Group, New York/London pp 68-163).

Customary bisphosphonate dosage forms, e.g. for the treatment of TIH, BM and MM, are intravenous infusion solutions. However, bisphosphonates solutions, although intrinsically stable, react with di- and polyvalent cations, especially calcium, barium, magnesium, aluminium, boron, and silicon present in glass to form insoluble precipitates giving rise to turbidity and possible loss of potency, neither of which can be tolerated in a pharmaceutical product. Further such precipitates may lead to blockage of blood vessels and thus could cause a thrombosis as serious complication of the medication. Thus long term storage of bisphosphonate solution formulations in standard glass vials, even of hydrolytic resistance class I quality is not possible. Also such solution in glass products cannot be terminally moist heat sterilized, and must be aseptically filled, because the leaching of cations is accelerated under the elevated temperature conditions of moist heat sterilization. It has been shown that at pH values acceptable for parenteral delivery, significant amounts of ions are leached out of commercially available glass containers (Farm. Vestnik. Vol 54, p. 331 (2003)). Consequently, for short term storage of solution in glass products it would be necessary to aseptically fill the solutions, although in view of their high chemical stability heat sterilisation of bisphosphonate solutions is inherently possible. Such aseptic filling does not comply with the currently accepted processing norms, as outlined in the document no. CPMP/QWP/054/98 corr., “Decision trees for the selection of sterilisation methods” issued by the European Agency for the Evaluation of Medicinal Products (EMEA). The same document also states that “the use of an inappropriate heat-labile packaging material cannot be in itself the sole reason for adoption of aseptic processing”.

Consequently bisphosphonate products for iv infusion are typically provided in the form of solid lyophilisates, which do not show microbial growth promoting properties when compared with unpreserved bisphosphonate solutions at physiologically acceptable pHs. The lyophilisates are made up into the infusion solution with water for injection or other aqueous solvents shortly before use. Commercial drug products that are processed as described above are sold under the trade name of e.g. Aredia® and Zometa®. In view of the low solubility of the precipitates formed with divalent and polyvalent cations, even the low levels of alkaline earth metal impurities present in all commercially available grades of sodium chloride and saline solutions could result in formation of such precipitates when diluting concentrated bisphosphonic acid solutions.

Recently it has been proposed (WO 02/22136, F. H. Faulding & Co Ltd.) to provide a pharmaceutical product comprising a container containing a diphosphonate in solution, wherein the solution: (a) has a pH of between 5 and 8; and (b) is free of organic buffer and polyethylene glycol and wherein the container is a glass container in which the surface in contact with the solution has been pre-treated to protect against leaching of impurities from the glass by the solution or wherein the container consists of at least one component manufactured from a non-glass material, such as polyethylene, polypropylene and polymethylpentene. However, WO 02/22136 does not include any teaching as to how, when or if the product is sterilised. Further this reference does not give guidance on how to keep the pH value stable over storage time if highly potent low dosed bisphosphonates as e.g. zoledronic acid is formulated.

It has now been found that bisphosphonate solutions may be formulated for long term storage in containers comprising polymeric materials which containers do not chemically interact with the bisphosphonate solution and which may be conveniently terminally sterilised.

### BRIEF DESCRIPTION OF DRAWINGS

FIG. 1 shows endotoxin reduction rate in Dalkyo CZ plastic vials on standard washing machines.

### DETAILED DESCRIPTION OF INVENTION

Accordingly the present invention provides a bisphosphonate solution pre-concentrate pharmaceutical product comprising a container containing a bisphosphonate solution, in which at least the internal surface of the container comprises a plastic material and in which the container is heat sterilisable.

The products of the present invention are advantageously solution products for parenteral administration which do not require reconstitution of a lyophilisate prior to use. Conveniently also the product may be heat sterilised in situ in the container during production, preferably terminally moist heat sterilised (e.g. by steam thus advantageously obtaining a Sterility Assurance Level of at least 10⁻⁶). Additionally, these solutions may be diluted using commercially available infusion media (e.g. Water for Injection, normal saline solution or isotonic glucose solution) without the risk of precipitation of insoluble complexes of the bisphosphonic acid with earth alkaline metal impurities.

The products of the invention may be administered orally, transdermally, or by injection, e.g. subcutaneously, arterially or intravenously. Most preferably the products of the invention are administered by intravenous infusion.

The products of the invention comprise solutions which require dilution before administration and as such are referred to as “solution pre-concentrates”.

Preferably the solution pre-concentrate product is in the form of a unit dose solution pre-concentrate, i.e. contains sufficient bisphosphonate for a single dose treatment. Such unit dose solution pre-concentrate products typically have a volume in the range from about 1 ml up to about 20 ml, preferably in the range from about 2 ml up to about 10 ml, most preferably about 5 ml, e.g. 4 ml, 5 ml or 6 ml (wherein such volumes may additionally include up to 0.5 ml, e.g. about 0.2 ml or about 0.3 ml, overfill to accommodate for liquid remaining in the container when the solution pre-concentrate is withdrawn for dilution.).

The solution pre-concentrate is diluted, e.g with water for injection (WFI), 5% dextrose solution, 0.9% sodium chloride solution or any other solution free of di- and polyvalent cations, prior to use, typically to a final volume in the range from about 20 up to about 300 ml, usually from about 50 to about 100 ml, preferably about 100 ml.

Such solution pre-concentrates typically comprise a pH adjusting agent, preferably a basic pH adjusting agent, more preferably an organic base. It further has been found that compared to strong inorganic bases as sodium hydroxide, the organic bases are able to form in situ a slight buffering system with the bisphosphonate itself which enables more easily adjustment of the desired pH-value and ensures optimal stability of the pH value over the whole storage time. The pH of the solution pre-concentrate is preferably in the region from about pH 4.5 up to about pH 8, more preferably in the range from about pH 5.5 up to about pH 7.5, e.g. about pH 6.3 or about pH 6.5 or about pH 6.8 or about pH 7.2. Examples of suitable organic bases include the sodium or potassium salts of organic acids as acetic acid, citric acid, lactic acid, glutamic acid, tartaric acid, fumaric acid, maleic acid, or malic acid. Furthermore, basic forms of amino acids may be used, e.g. histidine or arginine. Examples of suitable anorganic bases are sodium or potassium phosphate, sodium hydrogen carbonate or sodium hydroxide. Also mixtures of the above bases, or mixtures of the bases with their corresponding acids may be used. For example, the formulation may comprise a base, e.g. sodium citrate, with an acid, e.g. hydrochloric acid. Preferably the base is a sodium or potassium salt. When using potassium salts, the physiological tolerability of such formulations however have to be carefully assessed, and it is recommended not to exceed in the final diluted infusion solution the physiological concentration of potassium in blood serum which is approx. 4 milli-moles per litre.

Preferably the pH-adjusting agent is a salt. More preferably the salt is selected such that the complexing constant of the salt with di- and polyvalent cations is higher than the complexing constant of the bisphosphonic acid with such cations, advantageously to avoid precipitation of insoluble complex salts after dilution with commercially available diluents which often contain traces of such cations.

Such solution pre-concentrates may also typically comprise an isotonising agent. Preferably the tonicity of the solution pre-concentrate is in the range from about 200 mOsm/kg up to about 500 mOsm/kg, more preferably from about 250 mOsm/kg up to about 350 mOsm/kg, e.g. about 280-300 mOsm/kg. Examples of suitable nonionic isotonising agents are: glycerol, polyethylene glycol, propylene glycol, ethanol, cyclodextrins, amino acids, sugars and sugar alcohols including: Glucose, fructose, mannose, mannitol, saccharose, lactose, trehalose, maltose, sorbitol. Suitable ionic isotonising agents are sodium chloride, sodium nitrate, potassium chloride, ammonium chloride.

Preferably the isotonising agent is a non-ionic isotonising agent, more preferably a sugar, ester, alcohol or polyol. Particularly preferred isotonising agents for use in the solution pre-concentrate are mannitol, 1,2 propylene glycol, glycerol and sorbitol, of which mannitol is particularly preferred.

Another preferred variant is the use of ionic isotonising agents, of which sodium chloride is particularly preferred.

Conveniently, the solution pre-concentrate may also contain an excess of isotonizing agent such that the solution becomes isotonic after dilution to the final infusion volume with water for injection.

Thus in a preferred embodiment the invention provides a solution pre-concentrate pharmaceutical product comprising a container containing a bisphosphonate solution comprising

a) a unit dose of a bisphosphonate;

b) an organic base, and

c) an isotonising agent

in which at least the internal surface of the container comprises a plastic material and in which the filled container is terminally heat sterilisable.

The container for the product of the invention may comprise a glass container having a transparent plastic inner lining. Preferably, however, the container is made of plastic material and does not comprise a glass outer shell. Examples of plastic materials which may be used include: polysulfone, polycarbonate, polypropylene, polyethylene (LDPE or HDPE), ethylene/propylene copolymers, polyolefines, acrylic-imide copolymers, PVC, polyester (e.g. PET, PEN and the like), Teflon, Nylon, acetal (Delrin), polymethylpentene, PVDC, ethylvinylacetate, AN-copolymer etc. The plastic material used for either type of container is preferably a transparent plastic material, i.e. it is translucent and permits visual inspection of the contents.

Furthermore the plastic material used is a plastic which is capable of withstanding heat sterilisation in the filled and unfilled state, preferably moist heat sterilisation e.g. steam sterilisation or superheated water showering sterilisation, at a temperature of at least about 110° C. to about 130° C. or higher, e.g. at a temperature of at least 121° C., e.g. at 121-124° C.

Particularly preferred is a completely transparent uncoloured plastic material that is clear like glass, e.g. polycarbonate, polysulfone, cycloolefinic polymers such as Dalkyo CZ resin, thermoplastic olefin polymers of amorphous structure (e.g. TOPAS, manufactured by Ticona). Most preferred are Dalkyo CZ resin and similar cycloolefinic polymers.

Solution pre-concentrate products may be provided in plastic or plastic-coated vials, typically having a volume from about 2 ml up to about 20 ml, e.g. about 5 ml.

Bisphosphonate solutions may also be administered by slow intraveneous injection of a more concentrated form, e.g. with a concentration in the range from about 0.01 to about 0.5, more usually from about 0.05 up to about 0.2 mg bisphosphonate/ml. For this purpose the product may also be filled into prefillable syringes that can be terminally moist heat sterilized, e.g. in syringes made of Dalkyo CZ resin or similar or of thermoplastic olefin polymers of amorphous structure (e.g. as sold by Schott under the trade name Schott Top Pac or similar)

Commercially available plastic container materials like the Dalkyo CZ resin further have a thermal deformation temperature according to ASTM D648 of 123° C., which would narrow down the acceptable sterilization temperature to at most 123° C. It has now been found that sterilization even at significantly higher temperatures of e.g. up to 130° C., lead neither to measurable deformations of the container nor to impaired container closure integrity.

Preferably the bisphosphonates for use in the invention are the nitrogen containing bisphosphonates, including those having side chains which contain amino groups or especially those having side chains containing nitrogen-containing heterocycles, most especially containing aromatic nitrogen-containing heterocycles.

Examples of suitable bisphosphonates for use in the invention may include the following compounds or a pharmaceutically acceptable salt thereof: 3-amino-1-hydroxypropane-1,1-diphosphonic acid (pamidronic acid), e.g. pamidronate (APD); 3-(N,N-dimethylamino)-1-hydroxypropane-1,1-diphosphonic acid, e.g. dimethyl-APD; 4-amino-1-hydroxybutane-1,1-diphosphonic acid (alendronic acid), e.g. alendronate; 1-hydroxy-ethidene-bisphosphonic acid, e.g. etidronate; 1-hydroxy-3-(methylpentylamino)-propylidene-bisphosphonic acid, ibandronic acid, e.g. ibandronate; 6-amino-1-hydroxyhexane-1,1-diphosphonic acid, e.g. amino-hexyl-BP; 3-(N-methyl-N-n-pentylamino)-1-hydroxypropane-1,1-diphosphonic acid, e.g. methyl-pentyl-APD (=BM 21.0955); 1-hydroxy-2-(imidazol-1-yl)ethane-1,1-diphosphonic acid, e.g. zoledronic acid; 1-hydroxy-2-(3-pyridyl)ethane-1,1-diphosphonic acid (risedronic acid), e.g. risedronate, including N-methylpyridinium salts thereof, for example N-methylpyridinium iodides such as NE-10244 or NE-10446; 1-(4-chlorophenylthio)methane-1,1-diphosphonic acid (tiludronic acid), e.g. tiludronate; 3-[N-(2-phenylthioethyl)-N-methylamino]-1-hydroxypropane-1,1-diphosphonic acid; 1-hydroxy-3-(pyrrolidin-1-yl)propane-1,1-diphosphonic acid, e.g. EB 1053 (Leo); 1-(N-phenylaminothiocarbonyl)methane-1,1-diphosphonic acid, e.g. FR 78844 (Fujisawa); 5-benzoyl-3,4-dihydro-2H-pyrazole-3,3-diphosphonic acid tetraethyl ester, e.g. U-81581 (Upjohn); 1-hydroxy-2-(imidazo[1,2-a]pyridin-3-yl)ethane-1,1-diphosphonic acid, e.g. YM 529; and 1,1-dichloromethane-1,1-diphosphonic acid (clodronic acid), e.g. clodronate.

A particularly preferred bisphosphonate for use in the invention comprises a compound of Formula I

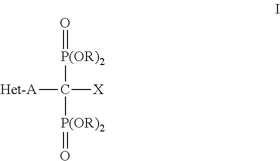

wherein
Het is an imidazole, oxazole, isoxazole, oxadiazole, thiazole, thiadiazole, pyridine, 1,2,3-triazole, 1,2,4-triazole or benzimidazole radical, which is optionally substituted by alkyl, alkoxy, halogen, hydroxyl, carboxyl, an amino group optionally substituted by alkyl or alkanoyl radicals or a benzyl radical optionally substituted by alkyl, nitro, amino or aminoalkyl;
A is a straight-chained or branched, saturated or unsaturated hydrocarbon moiety containing from 1 to 8 carbon atoms;
X is a hydrogen atom, optionally substituted by alkanoyl, or an amino group optionally substituted by alkyl or alkanoyl radicals, and
R is a hydrogen atom or an alkyl radical,
   and the pharmacologically acceptable salts thereof.

Examples of particularly preferred bisphosphonates for use in the invention are:
2-(1-Methylimidazol-2-yl)-1-hydroxyethane-1,1-diphosphonic acid;
2-(1-Benzylimidazol-2-yl)-1-hydroxyethane-1,1-diphosphonic acid;
2-(1-Methylimidazol-4-yl)-1-hydroxyethane-1,1-diphosphonic acid;
1-Amino-2-(1-methylimidazol-4-yl)ethane-1,1-diphosphonic acid;
1-Amino-2-(1-benzylimidazol-4-yl)ethane-1,1-diphosphonic acid;
2-(1-Methylimidazol-2-yl)ethane-1,1-diphosphonic acid;
2-(1-Benzylimidazol-2-yl)ethane-1,1-diphosphonic acid;
2-(Imidazol-1-yl)-1-hydroxyethane-1,1-diphosphonic acid;
2-(Imidazol-1-yl)ethane-1,1-diphosphonic acid;
2-(4H-1,2,4-triazol-4-yl)-1-hydroxyethane-1,1-diphosphonic acid;
2-(Thiazol-2-yl)ethane-1,1-diphosphonic acid;
2-(Imidazol-2-yl)ethane-1,1-diphosphonic acid;
2-(2-Methylimidazol-4(5)-yl)ethane-1,1-diphosphonic acid;
2-(2-Phenylimidazol-4(5)-yl)ethane-1,1-diphosphonic acid;
2-(4,5-Dimethylimidazol-1-yl)-1-hydroxyethane-1,1-diphosphonic acid, and
2-(2-Methylimidazol-4(5)-yl)-1-hydroxyethane-1,1-diphosphonic acid,
   and pharmacologically acceptable salts thereof.

More preferred bisphosphonates for use in the invention are Disodium-3-amino-1-hydroxy-propylidene-1,1-bisphosphonate pentahydrate (pamidronic acid) and 2-(imidazol-1yl)-1-hydroxyethane-1,1-diphosphonic acid (zoledronic acid) or pharmacologically acceptable salts thereof.

The most preferred bisphosphonate for use in the invention is 2-(imidazol-1yl)-1-hydroxyethane-1,1-diphosphonic acid (zoledronic acid) or a pharmacologically acceptable salt thereof.

Particularly preferred solution pre-concentrate products are in unit dose form and comprise from 1 to 10 mg of zoledronic acid or a pharmaceutically acceptable salt thereof. Most preferably the zoledronate unit dose product comprises an equivalent to 4 mg or 5 mg of anhydrous zoledronic acid, in particular as hereinafter described in the Examples.

Pharmacologically acceptable salts are preferably salts with bases, conveniently metal salts derived from groups Ia, Ib, IIa and IIb of the Periodic Table of the Elements, including alkali metal salts, e.g. potassium and especially sodium salts, and also ammonium salts with ammonia or organic amines.

Especially preferred pharmaceutically acceptable salts are those where one, two, three or four, in particular two or three, of the acidic hydrogens of the bisphosphonic acid are replaced by a pharmaceutically acceptable cation, in particular sodium, potassium or ammonium, in first instance sodium.

A very preferred group of pharmaceutically acceptable salts is characterized by having at least one acidic hydrogen and one pharmaceutically acceptable cation, especially sodium, in each of the phosphonic acid groups.

All the bisphosphonic acid derivatives mentioned above are well known from the literature. This includes their manufacture (see e.g. EP-A-513760, pp. 13-48). For example, 3-amino-1-hydroxypropane-1,1-diphosphonic acid is prepared as described e.g. in U.S. Pat. No. 3,962,432 as well as the disodium salt as in U.S. Pat. Nos. 4,639,338 and 4,711,880, and 1-hydroxy-2-(imidazol-1-yl)ethane-1,1-diphosphonic acid is prepared as described e.g. in U.S. Pat. No. 4,939,130. See also U.S. Pat. Nos. 4,777,163 and 4,687,767 and EP 0 275 821 B.

The invention also includes processes for the production of the solution products of the invention, which processes typically comprise a terminal heat sterilization step.

Accordingly in a further aspect the invention comprises a process for the production of a bisphosphonate solution pre-concentrate pharmaceutical product comprising a container containing a bisphosphonate solution, in which a bisphosphonate solution is provided within a container in which at least the internal surface of the container comprises a transparent plastic material and in which the container containing the bisphosphonate solution is terminally heat sterilised.

Thus the container containing the bisphosphonate solution is heat sterilized, preferably moist heat sterilised e.g. by saturated steam, steam/air mixtures or superheated water showering sterilisation, at a temperature of at least about 110° C. to about 130° C. or higher, e.g. at a temperature of at least 121° C. or higher, e.g. preferably at about 121-124° C. The effective sterilization time depends on the D-value of test spores in the solution and should be dimensioned that an overall Sterility Assurance Level of at least 10⁻⁶, preferably of at least 10⁻¹² is obtained. The effective sterilization time (dwell time) may be from about 15 minutes up to about 3 hours, conveniently from about 15 minutes to about 2 hours, e.g. preferably about 30-50 min. Advantageously the heat sterilisation is terminal heat sterilisation, i.e. heat sterilisation which is carried out near to or at completion of the production process, after filling of the container with the bisphosphonate solution and preferably after closure of the container, e.g. with a suitable cap, stopper or other closure. Conveniently standard production equipment for processing of glass vials may be used.

Suitable rubber stoppers are those which show only negligible leaching of metal ions like calcium, magnesium, zinc or silica when contacted with aqueous solutions, e.g. bisphosphonate solutions. Preferred stoppers have a low ash content and are coated on the product side with an impermeable and inert barrier, e.g. made of ETFE, Teflon or fluorinated elastomers. Suitable stoppers are e.g. Dalkyo D-777-1, Dalkyo D-777-3, Dalkyo D-713, Dalkyo D-21-7S, all coated on the product side with an ETFE layer, or Helvoet FM259/0 coated with a layer of a fluoropolymer (e.g. the Helvoet proprietary material Omniflex or Omniflex plus).

The bisphosphonate solution may be prepared in bulk and delivered to the containers; for instance, using the customary art procedures. The bulk bisphosphonate solution may be in the form of a solution of the free bisphosphonic acid, e.g. zoledronic acid, or in the form of a salt thereof, e.g. the sodium salt. Bulk bisphosphonate salt solutions may be prepared by dissolving the salt in aqueous media, or may be prepared in situ in solution by reaction of a dispersion of the free bisphosphonic acid with a base, e.g. neutralisation of the acid with sodium hydroxide to give the mono sodium salt, disodium salt, trisodium salt or tetra sodium salt as desired, e.g. disodium pamidronate or disodium zoledronate.

According to GMP requirements, all container material used for parenteral products are to be subjected to a depyrogenization process ensuring an endotoxin reduction of at least 3 log units. Heat depyrogenisation is customarily used for glass vials. However, plastic vials generally cannot be processed on standard pharmaceutical sterile drug product filling lines, as such containers would not withstand the thermal stress applied in the heat depyrogenization tunnel. Therefore, plastic vials are usually processed without the necessary cleaning and depyrogenization steps, thus bearing the risk of contamination of the parenteral drug product with foreign matter present in the vials as well as with Endotoxins that may be dissolved from the vial material surface. Surprisingly it has been found in accordance with the present invention that some plastic containers can be processed on standard filling lines for glass vials, and that provided the washing process is suitably adjusted an endotoxin reduction by the factor of at least 1000 can be reproducibly obtained.

Thus in addition to the sterilisation step, the containers, in particular the plastic containers, may be depyrogenised prior to filling with bisphosphonate solution. We have found that washing of the plastic vials with water under pressure gives satisfactory depyrogenisation, e.g. reduction in endotoxin concentration by a factor of at least 1000 or more, e.g. about 16000-100000. Such a depyrogenisation step is preferably included within the production processes of the invention.

Alternatively endotoxin-free or substantially endotoxin-free plastic containers may be obtained from a supplier and such containers used without need for depyrogenisation.

The particular mode of administration and the dosage for the products of the invention may be selected by the attending physician taking into account the particulars of the patient, especially age, weight, life style, activity level, hormonal status (e.g. post-menopausal) and bone mineral density as appropriate. Most preferably, however, the bisphosphonate is administered intravenously.

Normally the dosage is such that a single dose of the bisphosphonate active ingredient from 0.002-20.0 mg/kg, especially 0.01-10.0 mg/kg, is administered to a warm-blooded animal weighing approximately 75 kg. If desired, this dose may also be taken in several, optionally equal, partial doses.

“mg/kg” means mg drug per kg body weight of the mammal—including man—to be treated.

Preferably, the bisphosphonates are administered in doses which are in the same order of magnitude as those used in the treatment of the diseases classically treated with bisphosphonic acid derivatives, such as Paget's disease, tumour-induced hypercalcemia or osteoporosis. In other words, preferably the bisphosphonic acid derivatives are administered in doses which would likewise be therapeutically effective in the treatment of Paget's disease, tumour-induced hypercalcaemia or osteoporosis, i.e. preferably they are administered in doses which would likewise effectively inhibit bone resorption.

The following Examples illustrate the invention described hereinbefore.

### EXAMPLES

### Example 1

### Zoledronic Acid 4 mg/5 mL

| Ingredient | Amount [kg] per 500 L |
|---|---|
| Zoledronic acid monohydrate | 0.4264 kg |
| Corresponding to 0.400 kg | |
| zoledronic acid anhydrous | |
| Mannitol | 22.00 kg |
| Sodium citrate | 2.400 kg |
| Water for injection | Up to 507.5 kg = 500 L |

Approx. 85-95% of the total amount of water for injection is filled into a stainless steel compounding vessel. The excipients mannitol and sodium citrate are added and dissolved under stirring. The drug substance zoledronic acid is added and dissolved under stirring. The preparation is adjusted to the final weight with water for injection. The bulk solution is passed to the filling line and filtered in-line through a filter of 0.2 μm pore size. Washed and dried 5 mL Dalkyo CZ plastic vials are filled with 5.3 ml of bulk solution. Sterilized Helvoet FM259/0 Omniflex plus coated stoppers are inserted into the vials, and the stoppered vials are sealed with aluminium caps. The vials are sterilized with moist heat to obtain a Sterility Assurance Level of 10⁻¹², i.e. at 121-123° C. for 41 minutes (effective dwell time). As can be seen from the table below, the drug product does not show any sign of degradation and is stable at room temperature for at least 24 months.

| | | | | 40° C./75% rel | |
|---|---|---|---|---|---|
| | | | 30° C./70% rel. | humidity | |
| | | | humidity | inverse storage | 50° C. |
| Test | | Start | 24 months | 6 months | 1 month |
| Assay | | 99.8% | 101.4% | 100.7% | 101.0% |
| Degradation | | 0.1% | 0.1% | 0.2% | 0.2% |
| products, sum | | | | | |
| pH-value | | 6.3 | 6.3 | 6.3 | 6.3 |
| Particulate | ≧10 μm | 17 | 18 | 4 | not |
| matter (USP) | ≧25 μm | 2 | 1 | 0 | determined |

| | | | | |
|---|---|---|---|---|
| Appearance | clear, | clear, | clear, | clear, |
| | colorless | colorless | colorless | colorless |
| | solution | solution | solution | solution |
| Extractables | <0.2 μg/mL | <0.2 μg/mL | <0.2 μg/mL | <0.2 μg/mL |

### Example 2

### Zoledronic Acid 5 mg/5 mL

| Ingredient | Amount [kg] per 500 L |
|---|---|
| Zoledronic acid monohydrate | 0.533 kg |
| Corresponding to 0.500 kg | |
| zoledronic acid anhydrous | |
| Sodium chloride | 37.50 kg |
| Sodium citrate | 30.000 kg |
| Water for injection | Up to 503.0 kg = 500 L |

Approx. 85-95% of the total amount of water for injection is filled into a stainless steel compounding vessel. The excipients sodium chloride and sodium citrate are added and dissolved under stirring. The drug substance zoledronic acid is added and dissolved under stirring. The preparation is adjusted to the final weight with water for injection. The bulk solution is passed to the filling line and filtered in-line through a filter of 0.2 μm pore size. Washed and dried 5 mL Dalkyo CZ plastic vials are filled with 5.2 ml of bulk solution. Sterilized Helvoet FM259/0 Omniflex plus coated stoppers are inserted into the vials, and the stoppered vials are sealed with aluminium caps. The vials are sterilized with moist heat to obtain a Sterility Assurance Level of 10⁻¹², i.e. at 121-123° C. for 38 minutes (effective dwell time).
As can be seen from the table below, the drug product does not show any sign of degradation and is stable at room temperature for at least 36 months.

| | | | 25° C./60% rel. | 40° C./75% rel | |
|---|---|---|---|---|---|
| | | | humidity | humidity | 40° C. |
| | | | inverse storage | inverse storage | dry |
| Test | | Start | 36 months | 6 months | 6 months |
| Assay | | 100.8% | 99.0% | 102.7% | 102.6% |
| Degradation | | <0.1% | <0.1% | <0.1% | <0.1% |
| products, sum | | | | | |
| pH-value | | 6.0 | 6.0 | 6.0 | 6.0 |
| Particulate | ≧10 μm | 80 | not | 21 | 4 |
| matter (USP) | ≧25 μm | 2 | determined | 1 | 1 |
| Appearance | | clear, | clear, | clear, | clear, |
| | | colorless | colorless | colorless | colorless |
| | | solution | solution | solution | solution |
| Extractables | | <0.2 μg/mL | <0.2 μg/mL | <0.2 μg/mL | <0.2 μg/mL |

### Example 3

### Zoledronic Acid 8 mg/5 mL

| Ingredient | Amount [g] per 50 L |
|---|---|
| Zoledronic acid monohydrate | 85.28 g |
| Corresponding to 80 g of | |
| zoledronic acid anhydrous | |
| Mannitol | 1500.0 g |
| Sodium citrate | 480.0 g |
| Water for injection | Up to 50.75 kg = 50 L |

Approx. 85-95% of the total amount of water for injection is filled into a stainless steel compounding vessel. The excipients mannitol and sodium citrate are added and dissolved under stirring. The drug substance zoledronic acid is added and dissolved under stirring. The preparation is adjusted to the final weight with water for injection. The bulk solution is passed to the filling line and filtered in-line through a filter of 0.2 μm pore size. Each 5.2 mL are filled into plastic polypropylene containers in a Blow-Fill-Seal machine, e.g. a “bottlepack” machine type 314, 321 or 360 manufactured by Rommelag AG:
A plastic parison, extruded from Rexene 32M2 polypropylene (Huntsman), is accepted by an opened blow mould and cut below the die of the parison head. the main mould closes and simultaneously seals the bottom. A special mandrel unit settles onto the neck area and forms the parison into a container using compressed air or vacuum. Through the special mandrel unit, each 5.2 mL measured by the dosing unit is filled into the container. After the special mandrel unit retracts, the head mould closes and forms the required seal by vacuum. With the opening of the blow mould, the containers exits from the machine and the cycle repeats itself.
The plastic containers are sterilized with moist heat to obtain a Sterility Assurance Level of 10⁻¹², i.e. at 121-123° C. for 40 minutes (effective dwell time).

### Example 4

Zoledronic acid drug product manufactured according to example 1 is subjected to an additional sterilization for 60 minutes (effective) at 124.5° C., 126.5° C., 128.5° C. as well as 130.5° C. The vials shows no deformation of the most critical dimensions as the neck diameter and no change of the shape of the vial bottom which is shown to be most sensitive to heat stress damage. Container Closure Integrity is measured with 10 vials, by submerging into a 1% solution of methylene blue dye and treatment for 1 h at a pressure of 400 mbar followed by 1 h treatment at 1600 mbar. Any discoloration of the vial content due to ingress of the dye would be the sign for insufficient container closure integrity. All vials are tight according to this test.

| | Sterilization condition | | | | |
|---|---|---|---|---|---|
| | | 124.5° C., | 126.5° C., | 128.5° C., | 130.5° C., |
| | Untreated | 1 h | 1 h | 1 h | 1 h |
| Backpressure during | | 2.25 bar | 2.25 bar | 2.25 bar | 2.25 bar |
| cooling | | | | | |
| Change in inner | 12.49 mm ± | −0.04 mm | −0.04 mm | −0.05 mm | −0.07 mm |
| neck diameter | 0.02 mm | | | | |
| Container Closure | Complies | Complies | Complies | Complies | Complies |
| Integrity | | | | | |
| Shape of the | Concave | No | No | No | No |
| vial bottom | | deformation | deformation | deformation | deformation |

### Example 5

Zoledronic acid drug product manufactured according to example 1 is subjected to two additional sterilization cycles of each 60 minutes. No increase of particulate matter due to plastic material wear is found, no extractables is found in the solution, and no bacterial endotoxins that might leach out of the plastic vial polymer are accumulated in the solution.

| | | Number of cycles/cumulative dwell time | | |
|---|---|---|---|---|
| Test | | 1/40 min. | 2/100 min. | 3/160 min. |
| Appearance of the | | Colourless | Colourless | Colourless |
| container | | plastic vial | plastic vial | plastic vial |
| Particulate | ≧25 μm | 1 | 0 | 0 |
| matter | ≧10 μm | 41 | 14 | 10 |
| Extractables | Calculated as | <0.2 μg/ml | <0.2 μg/ml | <0.2 μg/ml |
| by HPLC | dibutyl- | | | |
| | phthalate | | | |
| Bacterial | | <0.4 EU/ml | <0.4 EU/ml | <0.4 EU/ml |
| endotoxins | | | | |

### Example 6

### Zoledronic Acid 8 mg/5 mL

| Ingredient | Amount [g] per 400 mL |
|---|---|
| Zoledronic acid monohydrate | 0.6822 g |
| Corresponding to 0.640 g | |
| zoledronic acid anhydrous | |
| Mannitol | 12.00 g |
| Tri-sodium phosphate 6AQ | 0.612 g |
| Water for injection | Up to 406.5 g = 400 mL |
| pH of the resulting solution | 6.5 |

### Example 7

### Zoledronic Acid 8 mg/5 mL

| Ingredient | Amount [g] per 400 mL |
|---|---|
| Zoledronic acid monohydrate | 0.6822 g |
| Corresponding to 0.640 g | |
| zoledronic acid anhydrous | |
| Mannitol | 12.00 g |
| Sodium hydroxide | 0.176 g |
| Water for injection | Up to 406.5 g = 400 mL |
| pH of the resulting solution | 7.0 |

### Example 8

### Zoledronic Acid 8 mg/5 mL

| Ingredient | Amount [g] per 400 mL |
|---|---|
| Zoledronic acid monohydrate | 0.6822 g |
| Corresponding to 0.640 g | |
| zoledronic acid anhydrous | |
| Mannitol | 12.00 g |
| Sodium tartrate dihydrate | 1.084 g |
| Water for injection | Up to 406.5 g = 400 mL |
| pH of the resulting solution | 4.7 |

### Manufacturing Process for Examples 6-8:

Approx. 85-95% of the total amount of water for injection is filled into a stainless steel compounding vessel. The excipients mannitol and sodium citrate are added and dissolved under stirring. The drug substance zoledronic acid is added and dissolved under stirring. The preparation is adjusted to the final weight with water for injection. The bulk solution is filtered in-line through a filter of 0.2 μm pore size. Washed and dried 5 mL Dalkyo CZ plastic vials are filled with 5.2 ml of the bulk solution. Sterilized Helvoet FM259/0 Omniflex plus coated stoppers are inserted into the vials, and the stoppered vials are sealed with aluminium caps. The vials are sterilized with moist heat at >121° C. for 20 minutes (effective dwell time).

### Example 9

### Zoledronic Acid Monohydrate 1 mg/5 mL

| Ingredient | Amount [g] per 100 mL |
|---|---|
| Zoledronic acid monohydrate | 0.0208 g |
| Sodium chloride | 0.6878 g |
| Sodium citrate dihydrate | 0.6832 g |
| Water for injection | Up to 100.0 mL |
| pH of the resulting solution | pH 6.8 |

Zoledronic acid, sodium chloride and sodium citrate are dissolved in approx. 60 mL of water for injection. The solution is filled up to 100.0 mL with water for injection. The solution is filtered through a 0.2 μm rated Pall Ultipor N66 filter. Each 5 mL of the filtrated solution are filled into steam sterilized Dalkyo CZ vials 5 mL/13 mm. The vials are closed with a Dalkyo D-713 13 mm serum bottle stopper and the stoppers are secured with an aluminum cap with plastic flip component. The vials are sterilized with steam for 20 minutes at 120° C.

| Test | before sterilization | after sterilization |
|---|---|---|
| pH-value | 6.8 | 6.8 |
| Osmolality | 280 mOsm/kg | 280 mOsm/kg |
| Assay | 98.0% | 97.8% |
| Imidazol derivative | 0.03% | 0.03% |
| Degradation products | 0.29% | 0.28% |
| Stopper extractable | <0.05% | 0.15% |
| (1-formyl piperidine) | | |

### Example 10

### Washing of Vials/Endotoxin Removal

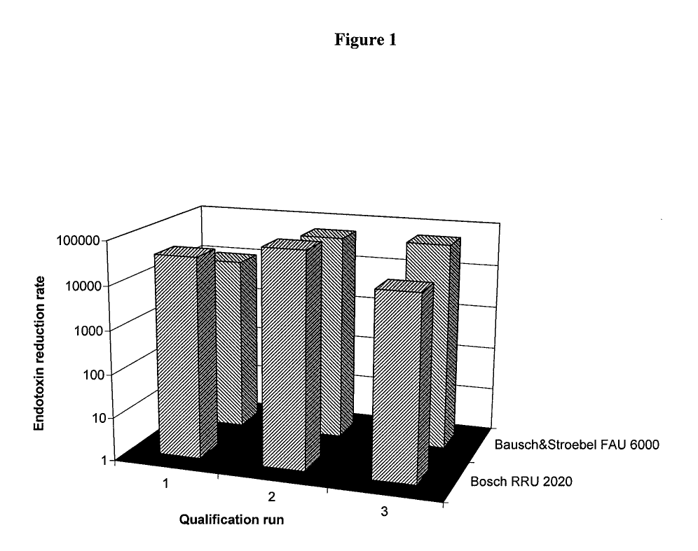

The plastic vials are processed on a conventional integrated automatic liquid filling processing line. Washing is performed in a conventional rotary vial washing machine (e.g. Bausch&Stroebel FAU 6000 or Bosch RRU 2020) as used for glass vials. The vials are put on the feeding belt of the washing machine. In a first instance the vials are submerged in a bath with hot water and treated by sonication. After that the vials are transported to the rotary washing station and are inverted. Cleaning is accomplished by a programmed process of air and water flushing through to nozzles inserted into the vials. The vials are first washed with recycled hot Water for Injections (>70° C.), blown out with filtered air, then washed again with fresh hot Water for Injections and blown out with filtered air. Following washing, the vials are inverted again to their normal position, and then transferred by the conveyor to the belt of the hot air tunnel, where they are dried at 110° C. On a Bausch&Stroebel FAU 6000 washing machine, a washing speed of 84 vials/min is suitable.
On a Bosch RRU 2020, a suitable washing speed is at a machine setting of 5.8-6.5 scale units The efficiency of this process is assessed by comparison of the endotoxin load of endotoxin-spiked vials prior and after the routine washing process. The results show more than 3 log reduction of the endotoxin challenge, i.e. the requirement of more than a 3 log reduction is met at each position tested during the washing process (see FIG. 1).

### Example 11

### Drying of Washed Vials in Hot Air

Drying of wet 5 mL Dalkyo CZ resin plastic vials is performed in a conventional hot air dryer. As a heat treatment of 125° C. for 10 hours does not show any significant influence on the vial dimensions, drying can be performed in a standard compact filling equipment line with the heat sterilization tunnel set at 110° C.

| | Untreated | 125° C., 10 h | 136° C., 1 h | 150° C., 1 h | 160° C., 1 h |
|---|---|---|---|---|---|
| Inner neck diameter | 12.49 mm ± | +0.01 mm | −0.10 mm | −0.22 mm | −0.33 mm |
| | 0.02 mm | | | | |
| Change in vial height | 38.61 ± | ±0.0 mm | ±0.0 mm | +0.2 mm | +0.1 mm |
| | 0.02 mm | | | | |
| Shape of vial bottom | Concave | Concave. | Slightly | Convex | Convex |
| | | No | convex | deformation | deformation |
| | | deformation | deformation | | |

### Example 12

### Compatibility of Zoledronic Acid 4 mg/5 mL (Example 1) with Infusion Solutions

5 ml concentrate for infusion is added to 50 ml infusion solution (sodium chloride 0.9% and glucose solution 5%). The first sample was withdrawn directly after mixing, a second sample is withdrawn after storage for 24 h at 2-8° C.
The chemical and physical stability is investigated. No change is observed for the quality characteristics appearance, assay and degradation products, s. table below.

Miscibility with 0.9% sodium chloride and 5% glucose solution

| | Sodium chloride solution 0.9% | | Dextrose solution 5%. | |
|---|---|---|---|---|
| | Start | 24 h | Start | 24 h |
| Appearance | Complies * | Complies | Complies | Complies |
| Assay | 99.6% | 99.4% | 99.2% | 99.2% |
| Degradation | <0.1% | <0.1% | <0.1% | <0.1% |
| products | | | | |

* clear as water

### Example 13

### Stability of Zoledronic Acid 4 mg/5 mL Upon Prolonged Sterilization Time

Vials containing zoledronic acid, formulated and packaged according to Example 1, are autoclaved in a steam autoclave at >121° C. for up to an overall 161 minutes (dwell time).
No decrease in the content of drug substance and no increase of degradation products is observed at an sterilisation times of up to 161 minutes. No release of leachables from vials or stoppers in amounts >0.2 μg/mL based on the UV-response of dibutylphthalate is observed.
The physical stability of the container material is supported by the fact that no increase of particulate matter is detected. Also no endotoxins in quantities above the LOQ of 0.4 EU/mL are released from the packaging material during this severe stress test.
The data show that there is no damage or deterioration of the product quality even after severe heat stress of 161 minutes sterilisation at >121° C.

| | Batch values | | | | | |
|---|---|---|---|---|---|---|
| | autoclaved | | autoclaved for an | | autoclaved for an | |
| | 41 min- | | additional 60 | | additional 120 | |
| Parameter | utes >121° C. | | min/>123° C. | | min/>123° C. | |
| Overall | 41 | min. | 101 | min. | 161 | min. |
| dwell time | | | | | | |
| at >121° C. | | | | | | |
| Appearance of | clear, | | clear, | | clear, | |
| the solution | colourless | | colourless | | colourless | |
| | solution | | solution | | solution | |
| Absorbance of | 0.00 | | 0.00 | | 0.00 | |
| the solution | | | | | | |
| pH value | 6.2 | | 6.3 | | 6.3 | |
| Particulate | | | | | | |
| matter | | | | | | |
| >25 μm | 1 | (USP | 0 | (USP | 0 | (USP |
| | 0 | (Ph. Eur.) | 0 | (Ph. Eur.) | 1 | (Ph. Eur.) |
| >10 μm | 41 | (USP), | 14 | (USP), | 10 | (USP), |
| | 52 | (Ph. Eur). | 12 | (Ph. Eur). | 11 | (Ph. Eur). |
| Extractables | <0.2 | μg/ml | <0.2 | μg/ml | <0.2 | μg/ml |
| by HPLC | | | | | | |
| Related | 0.1% | | <0.1% | | 0.1% | |
| substances, | | | | | | |
| by HPLC, sum | | | | | | |
| Assay of | 99.6% | | 99.6% | | 99.6% | |
| zoledronic | | | | | | |
| acid, by HPLC | | | | | | |
| Bacterial | <0.4 | EU/ml | <0.4 | EU/ml | <0.4 | EU/ml |
| endotoxins | | | | | | |

1. A pharmaceutical product in the form of a solution pre-concentrate comprising a container containing a bisphosphonate solution, in which at least the internal surface of the container comprises a plastic material and in which the container is heat sterilisable.

2. A product according to claim 1, in unit dose form having a volume of from about 1 ml up to about 20 ml.

3. A product according to claim 1, comprising a buffering agent.

4. A product according to claim 1, comprising an isotonising agent.

5. A pharmaceutical product comprising a container containing a bisphosphonate solution in the form of a solution pre-concentrate, comprising
d) a unit dose of a bisphosphonate;
e) an organic acid buffering agent, and
f) a non-ionic isotonising agent
in which at least the internal surface of the container comprises a plastic material and in which the filled container is terminally heat sterilisable.

6. A product according to claim 1, in which the container is a prefilled plastic syringe.

7. A product according to claim 1, in which the plastic material is transparent.

8. A product according to claim 1, in which the plastic material is a cycloolefinic polymer.

9. A product according to claim 8, in which the plastic material is a DAIKYO CZ resin or a similar cycloolefinic polymer

10. A product according to claim 1, in which the plastic material is a TICONA TOPAS polymer.

11. A product according to claim 1, in which the plastic material is a SCHOTT TOPPAC vial or syringe.

12. A product according to claim 1, in which the container is made by the Blow/Fill/Seal technology and the container material is selected from Polyethylene or Polypropylene.

13. A product according to claim 12, in which the container is made by the Blow/Fill/Seal technology and the container material is Polypropylene.

14. A product according to claim 13, in which the container is made by the Blow/Fill/Seal technology and the container material is Rexene 32M2 polypropylene.

15. A product according to claim 1, in which the bisphosphonate is an N-bisphosphonate or a pharmacologically acceptable salt thereof.

16. A product according to claim 15, in which the bisphosphonate is 2-(imidazol-1yl)-1-hydroxyethane-1,1-diphosphonic acid (zoledronic acid) or a pharmacologically acceptable salt thereof.

17. A process for the production of a pharmaceutical product comprising a container containing a bisphosphonate solution, in which a bisphosphonate solution is provided within a container in which at least the internal surface of the container comprises a transparent plastic material and in which the container is heat sterilised, preferably moist heat sterilised.

18. A process according to claim 17, in which the container is terminally heat sterilized.

19. A process according to claim 17, in which heat sterilization is at a temperature of at least about 110° C. to about 130° C.

20. A process according to claim 17, having a dwell time of from about 15 minutes up to about 3 hours.

21. A process according to claim 17, in which autoclaving conditions are applied to obtain a sterility assurance level of at least 10⁻⁶.

22. A process according to claim 17, in which autoclaving conditions are applied to obtain a sterility assurance level of at least 10⁻¹².

23. A process according to claim 17, in which the container is depyrogenised before filling with the bisphosphonate solution.

24. A process according to claim 17, in which an endotoxin/pyrogen-free or substantially endotoxin/pyrogen-free container is obtained and is filled with the bisphosphonate solution.

25. A process according to claim 17, in which the product comprises a non-ionic isotonising agent and in which ion chromatography, capillary electrophoresis, or high performance liquid chromatography is used for determination of the content of bisphosphonate and its by-products and degradation products.

26. A process according to claim 17, in which the product comprises a non-ionic isotonising agent and in which reversed phase chromatography is used for determination of the bisphosphonate and its by-products and degradation products.
